(19) United States
(12) Patent Application Publication
(10) Pub. No.: US 2023/0037437 A1
(43) Pub. Date: Feb. 9, 2023

(54) FORMULATIONS FOR USE IN THE TREATMENT OF DIFFERENT TYPES OF ADDICTIONS AND DEPENDENCE ON ADDICTIVE SUBSTANCES

(71) Applicant: UNIBIOLABS INTERNACIONAL S.A DE C.V, Puerto Peñasco (MX)

(72) Inventor: María Elena SELLES LEON, Puerto Peñasco (MX)

(21) Appl. No.: 17/787,510

(22) PCT Filed: Dec. 14, 2020
(86) PCT No.: PCT/MX2020/050057

§ 371 Date: Jun. 20, 2022

(30) Foreign Application Priority Data
Dec. 18, 2019 (MX) MX/A/2019/015582

(51) Int. Cl. A61K 31/195; A61K 31/198; A61K 31/7084; A61K 33/06; A61K 9/08; A61P 25/30

(52) U.S. Cl. CPC A61K 31/195; A61K 31/198; A61K 31/7084; A61K 33/06; A61K 9/08; A61P 25/30

(57) ABSTRACT

The present invention provides formulations comprising: L-threonine; Glycine; L-phenylalanine; DL-phenylalanine; Glutamine; NAD; and Magnesium, for use in the treatment of different types of addictions, such as: psychotropic medications, illicit drugs, tobacco and alcohol including, for example, but not limited to, narcotics, benzodiazepines, marijuana, cocaine, methamphetamines, antidepressants, and antipsychotics. The formulations of the present invention promote a detoxification process which facilitates that the brain repairs the damage caused by the abuse of addictive substances.

### FIELD OF THE INVENTION

The present invention belongs to the area of medicaments and pharmacological treatments that facilitate brain repair due to damage caused by the abuse of addictive substances, and specifically relates to formulations comprising amino acids, peptide, NAD and magnesium chloride (MgCl₂) for use in treating different types of substance addictions and addictive behaviors.

### BACKGROUND OF THE INVENTION

Various addictions are an emerging public health problem and high priority in the world.

Drug addiction is a chronic relapsing disease that is determined by alterations in the neurobiological mechanisms of brain function.

Chronic drug abuse is associated with a series of adaptive changes in brain physiology, in which physiological alterations progressively lead to addictive disorder (Goldstein, 2011, Nat Rev Neurosci.; 12(11): 652-669).

The abuse and dependence on psychoactive substances generates severe clinical problems, which is conditioned to the pattern of consumption, polydrug use, chronicity, the biological constitution of the subject and the early onset of consumption, for example, in adolescence, since the brain has not matured enough, making the user doubly vulnerable (https://www.samhsa.gov/disorders).

Substance use disorders lead to the development of brain disease that acutely alters its functioning as in the state of intoxication, which can be reversible but that in the face of a history of frequent intoxication syndromes alternated with withdrawal syndromes, cause a destabilization of the neurochemical functioning (neurotransmitters, receptors, transporters, etc.) of specific brain circuits of the reward area (ventral tegmental area, nucleus accumbens) and of the so-called global region composed of the hypothalamus, the septum, the amygdala, the ventral area of the olfactory nucleus, part of the cingulate area and the prefrontal cortex, which results in permanent and irreversible alteration of essential structures in brain function, triggering other psychiatric diseases, such as affect disorders, schizophrenia and other psychoses, simultaneously with substance dependence, which occurs in two out of three people with addiction (Kelly T M, Daley D C. Integrated treatment of substance use and psychiatric disorders. Soc Work Public Health. 2013; 28(0): 388-406).

Chronic drug use leads to tolerance and dependence, both psychological and physical, which causes a deterioration in impulse control and drug use, which when discontinued causes withdrawal syndrome, accompanied by the compulsive need to find and consume the drug (“craving”), in spite of knowing the harmful consequences that it may cause.

The Central Nervous System (CNS) plays an important role in the development of various addictions. Stimulation of neuronal receptors by these substances generates mechanisms of adaptation and response. At the synapse, the communication pathway between neurons, the electrical impulse becomes a chemical mediation caused by the release of substances, known as neurotransmitters, from nerve terminals.

Nevertheless, this is also the target site of action for many drugs that evoke a neurotransmitter-like response. The response caused by the action of the neurotransmitter, or by the substance on the receptor, can be of inhibitory or excitatory type, according to changes exerted in the membrane potential or ion channels. Currently, countless substances that gather the features of a neurotransmitter within the CNS are known, such as gamma-aminobutyric acid (GABA), glutamate, noradrenaline, dopamine, serotonin and peptides such as endorphins. A number of the latter were initially extracerebrally tested, but their action in the CNS has now been determined. Each one acts on a specific receptor and they are distributed in a defined way throughout the nervous system. They have been classified due to their cloning and the specific response to various substances.

As with other chronic diseases, relapses in drug use is more the rule rather than the exception, and this can occur during or after treatment, thus patients require prolonged treatment or several periods of treatment before being able to achieve sustained or long-term abstinence to obtain a fully restored of brain function.

Depending on its chemical structure, an addictive substance has the potential to cause the release of a particular substance, to stimulate specific receptors, to inhibit neurotransmitter transport mechanisms, or to stimulate multiple receptors. These characteristics have propitiated the pharmacological research in search of specific treatments for various addictions.

Neuroscientific research has led to the development of various pharmacological and behavioral treatments to treat substance dependence. Many have been successful, while some remain controversial on ethical grounds. The combination of drug therapy and behavioral therapy appears to be the most effective in treating dependence.

Regarding pharmacological treatments, one of the possibilities is represented by the use of substances or procedures in the area of neurotransmitters and receptors, which in one way or another, interfere with the action of the psychotic substance in the body, eliminating the positive reward provided by the consumption of the substance or causing it to become aversive, currently existing three types of biological treatments: substitution therapy, receptor blocking and vaccines.

For example, opioid receptor blockers (naloxone and naltrexone) reduce the rewarding effects of opioids and alcohol.

Another example is disulfiram, which interferes with the alcohol metabolism, creating aversion to its consumption. Also, disulfiram has been proposed as a treatment for cocainmania, since it prevents the metabolism of dopamine, a neurotransmitter that is released in high amounts when cocaine is consumed; excess dopamine results in symptoms such as anxiety, increased blood pressure, restlessness, and other symptoms. Compounds with effects similar to disulfiram have been discovered, such as coprine (N5-1-hydroxycyclopropyl-L-glutamine); temposil or citrated calcium carbamide, which has the same effects as disulfiram, but which is weaker and safer; chlorpropamide (first-generation sulfonylurea) used for the treatment of type II diabetes, which also causes an effect similar to disulfiram when consumed with alcohol. However, disulfiram should not be administered to patients taking certain drugs or antidepressants, as it also inhibits the enzyme dopamine-beta-hydroxylase, blocking the conversion of dopamine to norepinephrine. Combined with the antagonistic and/or anti-reuptake effect of stimulants, it can cause a dramatic increase in dopamine levels, resulting in insomnia, paranoia and in extreme cases psychosis.

Acamprosate (calcium acetyl homotaurinate) is another drug indicated in the maintenance therapy of abstinence in alcohol-dependent patients, since it has a stimulating activity of the neurotransmitter GABA (inhibitor) and an antagonistic action of neurotransmitter glutamate (exciter), thus restoring the normal activity of hyper-excited neurons as a result of chronic alcohol exposure. However, in preclinical studies, signs of toxicity related to excessive calcium intake, but not due to acetylhomotaurin, were detected. Alterations in phosphorus-calcium metabolism have been observed, including those causing diarrhea, soft tissue calcification, kidney and heart injuries.

Bupropion (chlorbutylketoamphetamine, a norepinephrine-dopamine reuptake inhibitor (NDRI) that blocks nicotinic receptors) is a drug with psychostimulant properties indicated as an antidepressant and which improves the abstinence rates of smokers, especially when combined with nicotine replacement therapy.

The use of vaccines that can prevent nicotine from acting on the brain has also been proposed. The vaccines have not yet entered the clinical trial phase, but studies in mice have provided promising results.

In the case of safe and effective treatments to help people stop using heroin or other opioids, there is the use of methadone (synthetic opioid agonist), which is usually given in liquid form in methadone maintenance programs (MMP), in a daily dose ranging individually from patient to patient according to their case. However, methadone causes pupil contraction, respiratory depression, bradycardia, muscle relaxation, antidiuretic hormone release, constipation, increased body temperature, and increased blood glucose concentration.

Buprenorphine (a derivative of thebaine, which has partial agonist activity of the mu (p) opioid receptor and weak antagonist of the kappa (κ) opioid receptor), has a relatively long action and has a good safety profile in the management of dependence on other opiates such as heroin.

In the case of levo-alpha-acetyl-methadol (LAAM, it is a synthetic opioid, mu (μ)-type opioid receptor agonist), it may be used in the treatment of heroin dependence. However, in Mexico, for example, acetylmethadol and its analogs are in group I of controlled narcotic substances.

Vanoxerine (GBR 12909) is a piperazine derivative that is a potent and selective dopamine uptake inhibitor (DRI). GBR-12909 binds to the target site on the dopamine transporter (DAT) approximately 50 times stronger than cocaine, but at the same time inhibits dopamine release. This combined effect only slightly raises dopamine levels, giving this drug only mild stimulant effects. GBR 12909 has also been shown to be a potent IKr channel blocker (hERG), and it also binds with nanomolar affinity to the serotonin transporter. However, the drug presented safety problems for its use as a cardiac antiarrhythmic.

In the case of immunotherapy against cocaine use, it is directed at the sequestration of cocaine in the bloodstreamby specific anti-cocaine antibodies, which prevent its entry into the brain.

In contrast to the drugs described above, amino acids are known to play a key role in almost all biological processes; the essential amino acids being those that favor the nervous connections and the neuroprotection of the brain, also benefiting the more autonomous functions such as respiration, temperature and the cardiovascular system.

Among the beneficial effects of essential amino acids in the brain it is found that:

They are involved in the process of absorption and metabolism of fatty acids, glucose and potassium, essential elements for proper functioning of the brain, helping the neurotransmitters that carry the necessary information to the brain connections so that they do their function correctly.

They act as channels for neurotransmitters to send information from the brain to the nervous system and spinal cord. They also serve to allow the nitrogen flows to the different organs that constitute the nervous system.

Moreover, substance abuse and aging are known to reduce the levels of NAD (nicotinamide adenine dinucleotide) in the body. This coenzyme is essential in all living cells, as it plays an important role in regulating mitochondrial energy production (electron and proton exchange and energy production of all cells). If the production of energy within nervous cells is being inhibited, not only difficulties in their function will occur, but also their repair will be difficult.

In this sense, the usefulness of NAD to increase the ability to concentrate, reduce chronic fatigue, stimulate metabolism, improve cardiovascular health, as well as to detoxify the body from alcohol, drugs and other substances that create dependencies has been proven. NAD+ plays an important role in reducing the effects of withdrawal syndrome by restoring the balance of neurotransmitters, which is usually altered when the use of the substance that causes addiction is stopped (Castro-Marrero J et al., 2015, and Humiston J., 2014).

Based on the importance of amino acids and NAD in brain function, there are compositions that prevent the development of pathological attraction to alcohol, and that have a protective effect against stress, which comprise (in mg/g): leukoanthocyanins 219-270, catechols 153-187, flavonols 81-99, lignin 68-83, reducing sugars 216-264, pectin 18-22, organic acids 76.5-93.5, sterols 4.5-5.5, methyl sterols 1.35-1.65, dimethyl sterols 1.98-2.42, lignans, lignan glycosides 9-11, phenolic aldehydes 4.5-5.5, alkylferulates 4.5-5.5; wherein said composition contains 27-33 mg of free amino acids (International Publication No. WO8911284 A1).

In addition, there are phytopharmaceutical food products that contain plant extracts and amino acids which may be used to reduce tobacco smoke addiction, and which contain up to 30% amino acids by weight, preferably between 13% and 17% (Publication of Patent Application No. US2002090441 A1).

Despite drug development for treatment of addictions, their effectiveness largely depends on the patients taking the medications. There is extensive experience indicating that the main problem with these drugs is treatment compliance. People with a long history of using a substance are often unable to keep the commitment to taking drug treatment on an ongoing basis.

For this reason, there is a need for novel formulations for use in the treatment of addictions and dependence on addictive substances, as well as in addictive behaviors, which do not have the technical drawbacks of the prior art, even if they are administered for a long time.

### SUMMARY OF THE INVENTION

The present invention is directed to formulations that provide the brain with specific nutrients, such as amino acids, peptides, magnesium and NAD (Nicotinamide Adenine Dinucleotide), which when properly combined result in pharmaceutical formulations useful in strengthening the brain and/or repairing brain damage caused by the abuse of psychoactive substances which generate a neuropsychological deterioration through different mechanisms of action.

The formulations of the present invention are indicated for the treatment of addictions and dependence on psychoactive substances, such as: psychotropic medications, illicit drugs, tobacco and alcohol, which include, for example, but are not limited to, narcotics, benzodiazepines, marijuana, cocaine, methamphetamines, antidepressants, and antipsychotics. Said formulations reduce or eliminate anxiety and restore mental clarity and function.

### DETAILED DESCRIPTION OF THE INVENTION

There are countless publications that note the effects of psychoactive substances in mammals, including humans. One of these publications is the one entitled “Neuroscience of psychoactive substance use and dependence” (2005), published jointly by the Organization of American States, the Inter-American Drug Abuse and Control Commission (CICAD, for its acronym in Spanish) and the World Health Organization, where it is stated that 8.9% of the total burden of morbidity is attributed to the use of psychoactive substances. Said document describes in detail the brain mechanisms: 1) Neurobiology and Neuroanatomy (Chapter 2), Biobehavioural Processes Underlaying Dependence (Chapter 3), and Psychopharmacology of Dependence for Different Drug Classes (Chapter 4).

In chapter 4, the document explains the effects on behavior, mechanisms of action of different drugs, tolerance, abstinence to the same, neurobiological adaptations to prolonged use and pharmacological treatment of dependence on different drugs.

Therefore, in the present invention, details already described regarding the psychoactive substances that are evident in light of the aforementioned document will be omitted and only reference will be made to aspects that are considered relevant to the invention.

In the present invention, it should be understood as a psychoactive substance or drug, unless indicated otherwise or it should be taken insofar as it favors a good understanding of the invention, any substance that, introduced into the body, by any route of administration, produces an alteration in the functioning of Central Nervous System and is susceptible to create dependence, either psychological, physical or both. In addition, psychoactive substances have the ability to modify consciousness, mood or thought processes of the subject who consumes them. (World Health Organization).

The terms and concepts used in the present patent application are described below.

Drug abuse, or drug addiction, means the frequent consumption of narcotics, despite knowing the negative consequences they produce. Among other things, narcotics modify brain functioning and its structure, causing dangerous behaviors. It is considered addiction, because it is difficult to stop using said substances, since they cause brain alterations in the regulatory mechanisms of decision-making and inhibitory control and because the user thereof spends a great amount of time in the search and consumption of the same (http://www.imss.gob.mx/salud-en-linea/adicciones). There is a large group of substances that result in more or less intense addiction, which are mainly characterized by: compulsive use (intake), craving, and withdrawal syndrome (when the administration is stopped). The number of substances for which a more or less intense dependence has been noted is relatively high and is constantly growing. These include opiates (morphine, heroin, etc.), psycho-dysleptics (LSD), alcohol, cannabinoids, and psychostimulants.

Addictive behaviors: any activity, substance, object or behavior that has become the main focus of a person's life excluding other activities, or that has begun to harm the individual and others physically, mentally or socially. It's not only about addictive behavior in relation to substances, the most socially recognized addiction, but it also includes behaviors and objects.

Physical addiction occurs at brain sites where neurons create the need for compulsive use, because the body has become used to the drug.

Psychological addiction is the need to consume a substance, which manifests itself at the level of thoughts or emotions, in the face of a stressful situation, or any problem. Therefore, there is no physical dependence, because receptors do not develop at the neuronal level for the action of the addictive substance.

Based on pharmacological elements (features), drugs are classified as:

Euphoric (euphoriants). Opium and derivatives, as well as cocaine.

Fantastical (hallucinogen). Mescaline, marijuana and henbane, among others.

Intoxicating. Alcohol, ether, chloroform and benzine.

Hypnotics. Barbiturics and other sleep inducers.

Excitatory (stimulants). Caffeine and tobacco (nicotine) among others.

New drugs or design drugs. These are created for recreational purposes. Some examples are: phenylethylamines, arylhexylamines, opioids, fentanyl derivatives, meperidine, and methaqualone.

Psychostimulants. These are a group of substances with diverse chemical structures and different mechanisms of action, which results in a strong activation of some CNS processes. All psychostimulants induce addiction in some extent, but their intensity is very different. The most used stimulants are cocaine and derivatives, amphetamines, caffeine, and nicotine.

Opioids (heroin, diacetylmorphine or diamorphine as its common international designation), is an opioid with analgesic properties which is also less commonly used as cough suppressor and antidiarrheal. Because of its euphoriant effects, heroin is used as a semisynthetic recreational drug. It is derived from morphine and obtained from the poppy, a plant from which opium is extracted. Its frequent and regular administration is associated with tolerance and a strong physical dependence, which are factors that motivate a compulsive use and abuse. Physical dependence develops from higher doses of this drug. In physical dependence, the body is adapted to the presence of the drug and withdrawal symptoms arise if drug administration decreases abruptly (Morán Chamorro I, Baldirá Martinez de Irujo J, Marruecos-Sant L, Nogué Xarau S. Intoxicación por opiáceos y opioides. In: Difusión G, editor. Toxicologia clinics. Madrid; 2011. p. 483-95).

Cocaine or benzoylmethylecgonine as per its common international designation, also known simply as coca, is a crystalline tropane alkaloid and powerful stimulant used mainly as a recreational drug. The most common ways of use are inhalation, insufflation, or injection in vein. The mental effects include loss of contact with reality, aggressiveness, increase of alertness, feelings of persecution, intense sensation of happiness, and psychomotor anxiety. The physical symptoms are fast heart rate, sweatiness and pupil dilation, and higher doses may cause increase of blood pressure, and increase of body temperature. Effects begin in seconds (or minutes) after use and last from five to ninety minutes. Although most use is illegal, cocaine has a small number of accepted medical uses such as topical anesthetic and anti-hemorrhagic during nasal surgery, among others.

Caffeine is an alkaloid compound of the xanthine group that is present in several plants such as coffee and cacao beans, tea leaves, guarana berries, and kola nuts. It is added to several soft drinks and medicines. Symptoms depend on endogen factors and the amount ingested.

Symptomatology is characterized by restlessness, nervousness, nervous excitement, insomnia, facial congestion, diuresis, gastrointestinal pain or discomfort, metabolic rate increased, muscular contractions, thinking and language disjointed, tachycardia or cardiac arrhythmia, periods of psychomotor activity or excitement (Morán Chamorro I, Baldirá Martinez de Irujo J, Marruecos-Sant L, Nogué Xarau S. Intoxicación aguda por bebidas energizantes (a base de taurina, inositol y cafeina). In: Difusión G, editor. Toxicologia clinics. Madrid; 2011. p. 535-8),there are data suggesting that caffeine causes a syndrome of clinical dependence very similar to that caused by other psychoactive substances (Ogawa N, Ueki H. Clinical importance of caffeine dependence and abuse. Psychiatry Clin Neurosci. 2007; 61:263-8). Caffeine has poor energy boost properties, but withdrawal syndrome is a reality and similar to that produced by other abused drugs. Caffeine blocks adenosine A1 and A2 receptors. Chronic administration of xanthines (caffeine, theophylline) causes a significant increase of adenosine, nicotine, and serotonin receptors in the brain. It also increases the number of L type calcium channels in neuronal membrane. Blocking of A1 and A2 receptors is necessary for the complete spectrum of pharmacological effects of caffeine. Adenosine receptors A1 and A2 are also involved in dependence to morphine and opioid withdrawal. Adenosine may reduce the dose dependence.

Amphetamine is a synthetic adrenergic agent and a powerful CNS stimulant. The term “amphetamines” has at least two possible meanings. The most restricted is used to refer to the triad formed by amphetamine, dextroamphetamine and methamphetamine, whereas the more general meaning also includes amphetamine-type stimulants (ATSs). ATSs are a pharmacological family formed by compounds having a chemical structure analogous to or derived from the amphetamine molecule, with similar clinical properties and a comparable level of pharmacological activity (potency). Stimulants such as methylphenidate (structural analogue), and dexmethylphenidate are also included in the group of amphetamine-type substances, as well as chemical derivatives having entactogen properties such as MDMA (ecstasy) and anorexigenic substances such as fenproporex, diethylpropion (amfepramone), phentermine, benzphetamine, and phendimetrazine, being these last two of lower relative potency.

Amphetamine is employed for therapeutic purposes and also as a recreational drug or to improve performance in sports. Amphetamine is a sympathomimetic agent highly potent in stimulating the CNS. The primary mechanism of action is the release of biogenic amines from their storage sites to the presynaptic areas of central synapses. The dependence and stereotyped behavior associated with amphetamine are induced by stimulation of dopamine release. Disturbances of perception and psychotic behavior may be due to serotonin and dopamine release in the mesolimbic system (Hoffman B B. Catecholamines, sympathomimetic drugs, and adrenergic receptor antagonists, In: Goodman & Gilman's The Pharmacological Basis of Therapeutics (Hardman J G, Limbird L E, eds.) Tenth International Edition, New York, Mc Graw Hill. 2001; 215-68).

Nicotine is an alkaloid found in the tobacco plant (Nicotiana tabacum) with high concentration in their leaves. This substance is also present in other plants of the Solanacea family. It is a powerful poison, and it has even historically used as insecticide. At lower concentrations this substance is a stimulant, which is the main factor of addiction to tobacco. It is a polar substance and soluble in water.

Alcohol, specifically ethanol, is a potent psychoactive drug with a high number of tertiary effects that may severely affect the body. The amount and circumstances of use have an important role when determining the duration of intoxication (Alhulia S. Intoxicación por drogas de abuso. Uninet. 2014). For example, alcohol ingestion after a big meal is less probable that causes visible signs of intoxication compared with ingestion in an empty stomach. Hydration plays also an important role when determining the duration of hangover. Alcohol-induced dependency is one of the most extended dependencies. Abuse and addiction depend, at least in part, on the activation of mesolimbic dopaminergic system. Activation of this system is made by ethanol directly, but also by acetaldehyde, which is derived from ethanol metabolism. Acetaldehyde increases the dopaminergic neuronal activity in the accumbens nucleus, the ventral tegmental area, and other sites of the CNS (Diana M, Peana A T, Sirca D, Lintas A, Melis M, Enrico P. Crucial role of acetaldehyde in alcohol activation of the mesolimbic dopamine system. Ann N Y Acad Sci. 2008; 1139:307-17; y Foddai M, Dosia G, Spiga S, Diana M. Acetaldehyde increases dopaminergic neuronal activity in the VTA. Neuropsychopharmacology. 2004; 29:530-6).

Marijuana or Cannabis contains THC (Delta-9-Tetrahydrocannabinol) which, when enter to the brain, makes the user feel euphoric because it acts on the reward center of the brain. This system is composed by the brain regions that rule the response of a person to pleasant things such as sex or chocolate, as well as most of abuse drugs. THC activates the brain's reward systemin the same way as almost drugs; this is, by stimulating brain cells to release dopamine (Morán Chamorro I, Baldirá Martínez de Irujo J, Marruecos-Sant L, Nogué Xarau S. Intoxicación aguda por derivados del cannabis. In: Difusión G, editor. Toxicologia clinics. Madrid; 2011. p. 463-7).

Marijuana use diminishes the ability to create new memories. Consumers of high doses of marijuana might experience an acute psychosis including hallucinations, delirium, and a loss of personal identity or self-recognition. Short term psychotic reactions to THC are different from the similar-to-schizophrenia alterations of longer duration associated to the use of cannabis in susceptible subjects. The damage to memory produced by marijuana occurs because THC modifies the way in which information is processed by hippocampus—the brain area in charge of memories formation.

Amino acids are organic molecules with an amine group (—NH₂) and a carboxyl group (—COOH) (Cambridge Dictionaries Online. “Amino Acid”. Cambridge University Press. 2015. Retrieved 3 July 2015; and, Nelson, David L.; Cox, Michael M., Principles of Biochemistry (4th ed.), New York: W. H. Freeman, (2005) ISBN 0-7167-4339-6).

Amino acids have important functions such as transmission of impulses in the nervous system, energy supply, and other functions.

Essential amino acids are those that cannot be synthetized by an organism and, therefore, have to be supplied by diet.

Aromatic amino acids include tryptophan and phenylalanine, which are the biosynthetic catalysts of neurotransmitters that help the brain to work properly. A diet high in proteins and magnesium influences the rapid absorption of aromatic amino acids in the brain, resulting in a more active brain with higher vitality.

Branched-chain amino acids include leucine, isoleucine, and valine, which are necessary for a good cerebral health and muscular strengthening. These are in charge of using the natural energy reserves of the body and the ingestion of these nutrients helps the brain to carry out its functions.

Threonine (Thr or T) is an essential amino acid with hydrophilic side chain (IUPAC-IUB Joint Commission on Biochemical Nomenclature “Nomenclature and Symbolism for Amino Acids and Peptides. 1983. Archived from the original on 9 Oct. 2008. Retrieved 5 Mar. 2018).

This is an amino acid that may be converted in blood glucose and hepatic glycogen by means of different paths. Accordingly, one of its main functions is the intervention for liver detoxification and a good performance of hepatic system. Threonine is metabolized to glycine.

Glycine (Gly or G) is the smallest amino acid and the only one that is not chiral among the 20 amino acids present in a cell. This amino acid can be a tranquilizing neurotransmitter in the brain, as it is an inhibitory neurotransmitter in the Central Nervous System (CNS) that acts on specific receptors in the brain stem, bone marrow and retina.

Moreover, glycine is an excitotoxic neurotransmitter that modulates the

N-methyl-D-aspartate (NMDA) receptor in the cerebral cortex. This NMDA receptor participates actively in the development of the nervous system, in cerebral plasticity, and also in degenerative processes (Avila Ariel, Laurent Nguyen, and Rigo Jean-Michel. Glycine receptors and brain development, Cell Neurosci. 2013; 7: 184).

Phenylalanine (Phe or F) is a neutral non-polar amino acid and is one of the most hydrophobic amino acids as it contains a benzene ring and, therefore, it is an aromatic amino acid. The brain uses this amino acid to produce noradrenalin (in addition to other chemicals such as dopamine and epinephrine). Noradrenalin is a substance that transmits signals between nerve cells to promote alertness, vitality, and mood. On the other hand, L-phenylalanine is found in the structure of neuropeptides such as somatostatin, vasopressin, melanotrophin, encephalin, adrenocorticotropic hormone (ACTH), angiotensin, P substance, and cholecystokinin.

DL-Phenylalanine (DLPA) is a 50:50 mixture of the two forms of phenylalanine, i.e., D and L-Phenylalanine (DLPA) (Friedman, M. Nutritional and toxicological consequences of food processing, New York, Plenum Press. 1991; p. 447-481), which functions in the body are to improve mood, decrease pain, to help memory, and facilitate learning. It has been used for the treatment of arthritis, depression, menstrual cramps, headache, obesity, Parkinson disease, and schizophrenia.

Glutamine (Gln or Q) is a non-essential amino acid that has a side chain similar to that of glutamic acid amide, except the carboxylic acid group of glutamic acid is replaced by an amide group. Its endogen production is very important and it increases in stress and catabolism increment situations, thus being considered as a stress marker (Bonet A, Grau T. La glutamina, un aminoácido casi indispensable en el enfermo critico (Glutamine, an almost indispensable amino acid the critical patient) . Med Intensiva. 2007; 31: 402-406; and Garcia de Lorenzo A. Glutamina, puesta al dia y controversias (Glutamine, up-to-date and controversy). Nutr Clin en Medicina. 2008; 1: 23-35). Glutamin is one of the few amino acid molecules that has two nitrogen atoms. This characteristic makes this molecule ideal to provide nitrogen for metabolic activities of the body. Glutamine biosynthesis in the body contributes to ammonia “cleansing” in some tissues (toxic at some concentrations), especially in the brain, where acts to make that ammonia is transported to other body sites. Because of its poor stability for management, it is preferably administered as glutamine dipeptide, and as a dipeptide with other amino acids (alanine, glycine) that maintain stability. These dipeptides are easily hydrolyzed by peptidase activity, which allows using glutamine practically at 100%. The most used dipeptide is L-alanyl-L-glutamine. Glutamine has effects strictly nutritional: nitrogen and energetic balance, and non-nutritional effects: immunomodulation and antioxidant. The clinical effects more commonly associated to L-alanyl-L-glutamine administration are: improving of nitrogen balance, improving of insulin resistance, less morbidity-mortality, and decreasing of infections.

B-Nicotinamide adenine dinucleotide (NAD), also known as nicotinamide adenine dinucleotide (abbreviated NAD+ for its oxidized form, and NADH for its reduced form), is an essential coenzyme formed by a dinucleotide (two nucleotides) bonded by phosphate groups, one of them adenine based, and the other a nicotinamide. This substance has an important role in regulating the mitochondrial production of energy (electron and proton exchange, and energy production in all cells). Brain tissue is highly dynamic in terms of electrical activity and energy demand. Thus, brain is the organ that uses more energy, and it uses high quantities of metabolic energy for information processing, which is based in the participation of two substrates: glucose and oxygen. Maintaining the brain metabolic activity is highly expensive and there are not sufficient reserves to maintain this high metabolic activity (Jaramillo-Magaña José J. Metabolismo cerebral. ANESTESIOLOGÍA EN NEUROCIRUGÍA. 2013; 36(1) 183-185). Drug abuse and ageing reduce NAD levels in the body. If energy production in nervous cells is being inhibited, there will be not only difficulties for functioning, but also reparation will be difficult. NAD use to increase concentration ability, reduce chronic fatigue, stimulate metabolism, improve cardiovascular health, as well as to detoxify alcohol, drugs, and other dependence-inducing substances from the body, has also been proved.

Magnesium (Mg). Magnesium ion is essential to all living cells. Magnesium in free state (as a metal) is not found in nature, but it forms part of many compounds mainly oxides and salts. It is involved in primitive biochemical processes such as photosynthesis and cell adhesion, it has activity as a regulator of ribosome structure, in membrane transport, protein and nucleic acids synthesis, in generating and transmission of nerve impulses, in muscular and cardiac contraction, as well as in oxidative phosphorylation (Aranda Pilar, Planells Elena y LLopis Juan. Magnesio. Scientific Communication: Art o Technique? Ars Pharmaceutica. 2000; 41(1): 91-100). Magnesium is an essential chemical element for human beings. Most of it is found in bones, and its ions have important rolls in the activity of many coenzymes and ATP depending reactions. It has also a structural roll, as Mg²⁺ ion has a stabilizing function in the structure of DNA and RNA chains. It participates in formation of neurotransmitters and neuromodulators, in neuronal repolarization, and muscular relaxation (the action thereof being important in cardiac muscle). Magnesium acts as an energizing and sedative in the body. Magnesium is used as a natural sedative as it maintains the energy balance in neurons and acts on nerve transmission. It is used to treat stress and depression, and also as a muscular relaxant (Kirkland Anna E., Sarlo Gabrielle L. and Holton Kathleen F. The Role of Magnesium in Neurological Disorders. Nutrients. 2018(10): 730). Magnesium reduces the intensity of addiction to opioids and psychostimulants (cocaine, amphetamine, nicotine, and others). It also reduces cocaine self-administration and reduces relapses of cocaine and amphetamine consumption. In heroin addicts, alcohol abusers, and other toxic maniacs, intracellular and blood concentration of magnesium is lower than in healthy subjects. One of the mechanisms that is thought magnesium reduces consumption of some highly addictive substances is the moderate stimulant effect on the reward system of the brain.

Magnesium and other bivalent cations are factors that may have influence on the intensity of addiction or withdrawal symptoms (Ruiz Martinez M, Gil Extremera B, Maldonaldo M D, Cantero-Hinojosa J, Moreno-Abadia V. Trace elements in drug addicts. Klin Wochenschr. 1990; 68:507-11).

There are data showing that magnesium decreases the addiction intensity to opioids, by means of magnesium acetate administration (0.5 mEq/kg/day) thus reducing the experimental physical dependence (Nechifor M, Chelarescu D, Miftode M. Magnesium influence on morphine induced pharmacodependence in rats. Magnes Res. 2004; 17:7-13). The intensity of withdrawal symptoms induced by naloxone was reduced even when magnesium administration was interrupted during the withdrawal period. Magnesium aspartate (732 mg/day) administration for 12 weeks in patients addicted to heroin was also beneficial (Daini S, Tonioni F, Barra A, Lai C, Lacerenza R, Sgambato A, Bria P, Cittadini A. Serum magnesium profile in heroin addicts according to psychiatric comorbidity. Magnes Res. 2006; 19:162-6, and Karakiewicz B, Kozielec T, Chlubek D, Noceri I, Starczewski A, Brodowska A, Laszczylnska M. Serum magnesium concentration in drug addicted patients. Magnes Res. 2007; 20:55-7). Magnesium may potentially reduce addiction intensity by a series of mechanisms such as glutaminergic, serotonergic, and adrenergic neurotransmitter systems, as well as by means of several neurological hormones.

In cocaine abusers, magnesium reduced the craving for this substance (Margolin A, Kantak K, Copenhaver M, Avant SSK. A preliminary controlled investigation of magnesium L-aspartate hydrochloride for illicit cocaine and opiate use in methadone maintained patients. J Addict Dis. 2003; 22:49-61). Cocaine craving scores were 78% lower in patients taking magnesium than in patients taking placebo. Magnesium also reduced cocaine self-administration in patients and cocaine consumption in rats (Kantak K M, Edwards M A, O'Connor T P. Modulation of the discriminative stimulus and rate altering effects of cocaine by competitive and noncompetitive N-methyl D aspartate antagonists. Pharmacol Biochem Behav. 1998; 59:159-69). In cocaine addicts, the plasma level of Mg²⁺ is higher than in heroin addicts (Tonioni F, Martinotti G, Barra A, Martinelli D, Autullo G, Rinaldi C, Tedeschi C, Janiri L, Bria P. Cocaine use disorders and serum magnesium profile, Neuropsychobiology. 2009; 53:159-64).

N-Methyl-D-Aspartate (NMDA) receptors are essential for cocaine action in brain. NMDA antagonists and some coupled ion channel blockers (like magnesium) may modify the cocaine effects (Kantak K M, Edwards M A, O'Connor T P. Modulation of the discriminative stimulus and rate altering effects of cocaine by competitive and noncompetitive N-methyl D aspartate antagonists. Pharmacol Biochem Behav. 1998; 59:159-69).

Magnesium has a moderate effect of stimulating the brain reward system. This effect constitutes the mechanism by which magnesium reduces the cocaine consumption (Nechifor M, Chelarescu D, Ciubotariu D. The influence of magnesium induced stimulation of the reward system. Magnesium Res. 2010; 23:41-7).

Chronic smoking reduces the serum magnesium level (Niemela J E, Cecco S A, Rehak N N, Elin R J. The effect of smoking on the serum ionized magnesium concentration is method dependent. Arch Pathol Lab Med. 1997; 121:1087-92); while magnesium administration decreses the number of smoked cigarettes as well as nicotine addiction (Nechifor M, Chelarescu D, Mândreci I, Cartas N. Magnesium influence on nicotine pharmaco dependence and smoking. Magnes Res. 2004; 17:176-81).

Magnesium can potentially reduce nicotine addiction by acting as a partial antagonist of calcium entry neurons, thereby decreasing glutamate release and glutamatergic transmission, which is stimulated by nicotine. It is also involved on the synaptic release of dopamine and other catecholamines. In addition, an increment in neuronal magnesium concentration produces a reduction in sodium concentration, which decreases the stimulant effect of nicotine on nicotine receptors. Likewise, the nicotine addictive effect is reduced by diminished the nicotine effect on GABA synthesis. It also enhance some of the GABA effects and diminish some effects of the excitatory amino acids in drug dependence.

Incresedintraneuronal Mg²⁺ concentration is thought to reduce the intensity of the psychostimulant addiction to amphetamine in some serotonin receptors. Evidence in favour is the fact that Li⁺ (which increases intracellular magnesium concentration) has an antagonist effect with amphetamine at the level of the nucleus accumbens (Gray J A, Moran P M, Grigoryan G, Peters S L, Young A M, Joseph M H. Latent inhibition: the nucleus accumbens connection revisited. Behav Brain Res. 1997; 88:27-34).

It is considered that magnesium could reduce the stimulating effect of ethanol on dopaminergic systems by directly reducing presynaptic release of dopamine, but also by decreasing acetaldehyde production.

There is important evidence implicating the endogenous opioids in the processes of reward and reinforcement (Gianoulakis C. Endogenous opioids and addiction to alcohol and other drugs of abuse. Curr Top Med Chem. 2009; 9:999-1001). Endogenous opioids such as morphine, induce an increse of dopamine concentration in the nucleus accumbens, which is considered the most important structure for drug addiction. This is considered a common effect for many drugs involved in abuse. Ethanol increases the release of p receptors, which seems to be important for ethanol addiction(Herz A. Endogenous opiod systems and alcohol addiction. Psychopharmacology (Berl). 1997; 129:99-111). Magnesium reduces receptor binding of morphine and other p receptor agonists (Méndez M, Leriche M, Calva J C. Acute ethanol administration differentially modulates mu opioid receptors in the rat meso accumbens and mesocortical pathways. Brain Res Mol Brain Res. 2001; 94:148-56, and Rodriguez F D, Bardaji E, Trayno Jr R. Differential effects of Mg2+ and other divalent cations on the binding of tritiated opioid ligands. J Neurochem. 1992; 591:467-72). Thus, magnesium could reduce the stimulation of dopamine synthesis produced by large quantities of opiod peptides which, in turn, are induced by ethanol in the nucleus accumbens and VTA.

It should be understood that the embodiment or embodiments shown in the present description are not limiting thereof and are only examples of its application.

The formulations of the instant invention are formulations adapted to be administered to the patient intravenously.

The formulations comprise:

i) L-threonine;

ii) Glycine;

iii) L-phenylalanine;

iv) D,L-phenylalanine;

v) Glutamine, preferably in the L-alanyl/L-glutamine dipeptide form;

vi) NAD; and

vii) magnesium, in the form of a magnesium salt, preferably magnesium chloride,

Wherein in a first embodiment, L-threonine is in the range of 0.5-3.0 mg, glycine is in the range of 0.5-3.0 mg, L-phenylalanine is in the range of 0.5-3.0 mg, D,L-phenylalanine is in the range of 1.0-5.0 mg, L-alanyl/L-glutamine is in the range of 1.0-5.0 mg, NAD is in the range of 200-1200 mg, and MgCl₂ is in the range of 300-600 mg.

In a second embodiment, formulation (F1) comprises L-threonine in the range of 1.0-3.0 mg, glycine in the range of 1.0-3.0 mg, L-phenylalanine in the range of 1.0-3.0 mg, D,L-phenylalanine in the range of 2.0-5.0 mg, L-alanyl/L-glutamine in the range of 2.0-5.0 mg, NAD in the range of 500-1000 mg and MgCl₂ in the range of 400-600 mg.

In a third embodiment, formulation (F2) comprises L-threonine in the range of 1.0-3.0 mg, glycine in the range of 1.0-3.0 mg, L-phenylalanine in the range of 1.0-3.0 mg, D,L-phenylalanine in the range of 2.0-5.0 mg, L-alanyl/L-glutamine in the range of 2.0-5.0 mg, NAD in the range of 700-1200 mg and MgCl₂ in the range of 400-600 mg.

In a fourth embodiment, formulation (F3) comprises L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 300-500 mg and MgCl₂ in the range of 400-600 mg.

In a fifth embodiment, formulation (F4) comprises L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 200-300 mg and MgCl₂ in the range of 300-600 mg.

In a sixth embodiment, formulation (F5) comprises L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 200-400 mg and MgCl₂ in the range of 300-600 mg.

In a preferred embodiment, the components of the formulations of the instant invention are added to a 500 mL bag of injectable 0.9% NaCl solution to be administered intravenously.

In other preferred embodiment, the formulations of the instant invention are administered along with intravenous or oral supplements such as vitamins, probiotics, and minerals commercially available.

Additionally, in other preferred embodiment, the formulations of the instant invention are administered before or during the support therapies that include Colon Hydrotherapy, and Manual Lymphatic Drainage (MLD).

In other preferred embodiment, the formulations of the instant invention facilitate the repairment of brain due to damage caused by the abuse of addictive substances selected from the group consisting of psychotropic medicaments, illegal drugs, tobacco, and alcohol, including narcotics, benzodiazepines, marijuana, cocaine, methamphetamines, antidepressants, and antipsychotics, among others.

In other preferred embodiment, the formulations of the instant invention may be administered to subjects with depression or brain fog with no significant history of drug use, or subjects never exposed previously to drugs, among others, in order to improve mental acuity (logical and mental clarity) by favoring the cerebral impulse.

Preparation of Formulations

Each of the amino acids, dipeptide and NAD used in the formulations were weighed and reconstituted in normal saline to obtain the concentration indicated for each patient, then the volume of MgCl₂ equivalent to the desired concentration was added and each component was added to a 500 ml bag of 0.9% NaCl for intravenous administration.

Clinical Study

An Open Clinical study was carried out, i.e., a clinical trial without a control group. 46 patients (32 men and 14 women) between 21 and 62 years old were studied, voluntarily admitted to the addiction unit for their detoxification. Diabetics, other known psychiatric pathologies, and chronic systemic diseases (there were no patients with liver damage, nephropathy, or thyroid dysfunction), patients under 18 years of age, and pregnant women (women in childbearing age had a negative blood test) were excluded.

As a criterion for inclusion was used patients addicted to illegal substances or with background of prolonged exposition or addiction. This includes alcohol, narcotics, benzodiazepines (including other hypnotics that function with GABA receptor), tobacco, marijuana, cocaine, methamphetamine, antidepressants, and antipsychotics. Other types of addictions were also included such as videogames, social media, and pornography, some patients diagnosed with abuse of one substance, and other patients having addiction to other substances.

The first requirement for addict patients to be treated with the formulations of the instant invention was the same as for any approach of addiction treatment, the preparation of individual patients and the desire to be clean and sober. Some necessary factors for the selection of candidates were evaluated, and the success probability of the treatment for each one of them. Among the factors considered was that patients were aware of the severe negative effects that drug consumption causes in their lives. In most of the cases, the motivating fact was that the patient put at risk the employment loss or to lose the possibility of continue education due to drug abuse, or loose the family. Another important factor was that the patient had abandoned, at least for a time, the drug abuse in the past, and that the patient had a responsible attitude with respect to addiction.

The importance of absolute abstinence of drug or alcohol use during the treatment, particularly those substances relevant for the abuse history of the patients was informed to them.

The medical condition of intoxication and withdrawal syndromes during use and withdrawal of the substance are different for each type of drug with respect to the amount used and duration of use. Accordingly, patients were administered five formulations, depending on the addiction to be treated.

The formulations of the instant invention were administered to each patient intravenously with slow dropping for about 6-8 hours a day, continuously.

Treatment for currently addict patients generally lasted between 10 and 15 days, preferably without interruptions. For patients treated for a significantly lower drug exposure, the treatment generally lasted five to ten days.

Results

It was observed that the formulations of the present invention safely, consistently and significantly improved the symptoms caused by drug use (psychotropic medications, illicit drugs, tobacco and alcohol, which include narcotics, benzodiazepines, marijuana, cocaine, methamphetamines, antidepressants, antipsychotics) or alcohol in patients.

The decrease in withdrawal symptoms such as anxiety, depression, emotional emptiness, cravings, agitation, emotional outbursts, tremors, digestive discomfort (the gastrointestinal system has its own extensive and highly developed nervous system that uses the same neurotransmitters as the brain), incoherence was assessed through interviews (qualitatively), at the end of treatment. In some cases, the formulations significantly reduced or eliminated these symptoms altogether, restoring well-being, hope, mental clarity, improving sleep, energy and appetite. In addition, most of the patients noticed some physical improvement within a few hours after starting treatment or at least at the end of the first day.

It was observed that, from the fifth day of treatment, 52% of all patients (that is, 24 patients) reported feeling better than in a long time, and reported feeling that mental clarity had returned. On day 8 or 10, 83% (that is, 38 patients) of the total patients reported feeling calm and noted full recovery of mental clarity, appetite and sleep. This result is only achieved by effectively supporting the repair of actual neurotransmitter dysfunction caused by mind-altering addictive substances.

Administration of the formulations of the instant invention as treatment for currently addict patients is enough if applied uninterruptedly between 10 and 15 days. In the case of patients treated for a significantly lower drug exposure, the treatment generally lasts five to ten days. According to the observations of the study described herein, if a patient is treated many days after the physiologically comforting period, he/she might feel fatigue or malaise, which is rapidly resolved by stopping the treatment. It is highly probable the occurrence of this due to the high concentration of amino acids, as they are not necessary anymore to repair body tissues because reparation has been done. This phenomenon was rarely observed (2 patients) and it is unlikely that it happens if patients are treated within the treatment period recommended, depending on the clinical response of each patient.

Evidence showed that in the specific case of patients addicted to benzodiazepines, even if the same treatment is applied (between 10 and 15 days), the duration and intensity of administration of the formulations of the instant invention often requires an increment of about three to five days.

The following table shows the results of patients with various addictions, when treated with the formulations of the present invention.

| | | | | | | | RESULTS OBTAINED ** | | | | | |
|---|---|---|---|---|---|---|---|---|---|---|---|---|
| | | | | | | | METABOLIC | | PHYSIOLOGIC | | COGNITIVE | |
| | | | | | | | TESTS | | TESTS | | TESTS | |
| | | | | FORMU- | TREAT- | DRIP | NO | | NO | | NO | |
| | | GEN- | | LATION | MENT | MODIFI- | CHANG- | CHANG- | CHANG- | CHANG- | CHANG- | CHANG- |
| PATIENT | AGE | DER | ADDICTION | USED | DAYS | CATION* | ES | ES | ES | ES | ES | ES |
| | | | | | | | | YES | | YES | | YES |
| | | | alcoholism | | | | | | | | | |
| | | | Marijuana | | | | | | | | | |
| 2 | 41 | F | Chronic | F2 | 14 | | | YES | | YES | | YES |
| | | | alcoholism | | | | | | | | | |
| | | | Tobacco | | | | | | | | | |
| | | | dependency | | | | | | | | | |
| 3 | 25 | F | Tobacco | F5 | 16 | | | YES | | YES | | YES |
| | | | dependency | | | | | | | | | |
| | | | Benzodiaze | | | | | | | | | |
| | | | pine | | | | | | | | | |
| | | | dependency | | | | | | | | | |
| 4 | 32 | M | Chronic | F2 | 12 | | | YES | | YES | | YES |
| | | | alcoholism | | | | | | | | | |
| | | | Tobacco | | | | | | | | | |
| | | | dependency | | | | | | | | | |
| | | | Marijuana | | | | | | | | | |
| | | | dependency | | | | | | | | | |
| 5 | 35 | F | Tobacco | F5 | 10 | | | YES | | YES | | YES |
| | | | dependency | | | | | | | | | |
| 6 | 48 | F | Cocaine | F4 | 5 | | | YES | | YES | | YES |
| | | | dependency | | | | | | | | | |
| 7 | 30 | M | Alcoholism | F2 | 14 | | | YES | | YES | | YES |
| | | | Tobacco | | | | | | | | | |
| | | | dependency | | | | | | | | | |
| | | | Drug | | | | | | | | | |
| | | | combination | | | | | | | | | |
| | | | dependency | | | | | | | | | |
| | | | (marijuana, | | | | | | | | | |
| | | | cocaine and | | | | | | | | | |
| | | | ecstasy) | | | | | | | | | |
| 8 | 23 | M | Tobacco | F5 | 10 | YES | | YES | | YES | | YES |
| | | | dependency | | | | | | | | | |
| 9 | 32 | M | Chronic | F2 | 12 | | | YES | | YES | | YES |
| | | | alcoholism | | | | | | | | | |
| 10 | 36 | M | Chronic | F2 | 10 | | | YES | | YES | | YES |
| | | | alcoholism | | | | | | | | | |
| | | | Tobacco | | | | | | | | | |
| | | | dependency | | | | | | | | | |
| 11 | 34 | F | Ludopathy | Fl | 5 | YES | X | | X | | | YES |
| | | | (Addiction | | | | | | | | | |
| | | | to casino | | | | | | | | | |
| | | | games) | | | | | | | | | |
| | | | Impulse | | | | | | | | | |
| | | | control | | | | | | | | | |
| | | | disorder | | | | | | | | | |
| 12 | 22 | F | Alcoholism | F2 | 16 | | | YES | | YES | | YES |
| | | | Tobacco | | | | | | | | | |
| | | | dependency | | | | | | | | | |
| | | | Drug | | | | | | | | | |
| | | | combination | | | | | | | | | |
| | | | dependency | | | | | | | | | |
| | | | (marijuana, | | | | | | | | | |
| | | | methamphe | | | | | | | | | |
| | | | tamines) | | | | | | | | | |
| 13 | 46 | F | Chronic | F2 | 14 | | | YES | | YES | | YES |
| | | | alcoholism | | | | | | | | | |
| 14 | 44 | M | Alcoholism | F2 | 15 | | | YES | | YES | | YES |
| | | | Tobacco | | | | | | | | | |
| | | | dependency | | | | | | | | | |
| | | | Drug | | | | | | | | | |
| | | | combination | | | | | | | | | |
| | | | dependency | | | | | | | | | |
| | | | (marijuana, | | | | | | | | | |
| | | | benzodiaze | | | | | | | | | |
| | | | pines) | | | | | | | | | |
| 15 | 57 | M | Chronic | F2 | 12 | | | YES | | YES | | YES |
| | | | alcoholism | | | | | | | | | |
| 16 | 31 | M | Anxiety | Fl | 5 | | | YES | | YES | | YES |
| 17 | 25 | F | Chronic | F2 | 10 | | | YES | | YES | | YES |
| | | | alcoholism | | | | | | | | | |
| 18 | 55 | M | Benzodiaze | F3 | 15 | | | YES | | YES | | YES |
| | | | pine | | | | | | | | | |
| | | | dependency | | | | | | | | | |
| 19 | 32 | M | Chronic | F2 | 12 | | | YES | | YES | | YES |
| | | | alcoholism | | | | | | | | | |
| 20 | 29 | M | Alcoholism | F2 | 15 | | | YES | | YES | | YES |
| | | | Tobacco | | | | | | | | | |
| | | | dependency | | | | | | | | | |
| | | | Drug | | | | | | | | | |
| | | | combination | | | | | | | | | |
| | | | dependency | | | | | | | | | |
| | | | (marijuana, | | | | | | | | | |
| | | | cocaine, | | | | | | | | | |
| | | | methamphe | | | | | | | | | |
| | | | tamine) | | | | | | | | | |
| 21 | 53 | M | Chronic | F2 | 14 | | | YES | | YES | | YES |
| | | | alcoholism | | | | | | | | | |
| 22 | 30 | M | Chronic | F2 | 12 | | | YES | | YES | | YES |
| | | | alcoholism | | | | | | | | | |
| 23 | 27 | F | Chronic | F2 | 11 | | | YES | | YES | | YES |
| | | | alcoholism | | | | | | | | | |
| 24 | 23 | M | Tobacco | F5 | 10 | | | YES | | YES | | YES |
| | | | dependency | | | | | | | | | |
| 25 | 28 | M | Chronic | F2 | 14 | | | YES | | YES | | YES |
| | | | alcoholism | | | | | | | | | |
| | | | Psycho stim | | | | | | | | | |
| | | | ulant drug | | | | | | | | | |
| | | | dependency | | | | | | | | | |
| | | | (ecstasy) | | | | | | | | | |
| 26 | 40 | M | Tobacco | F5 | 13 | | | YES | | YES | | YES |
| | | | dependency | | | | | | | | | |
| | | | Anxiety | | | | | | | | | |
| 27 | 26 | M | Chronic | F2 | 12 | | | YES | | YES | | YES |
| | | | alcoholism | | | | | | | | | |
| | | | Tobacco | | | | | | | | | |
| | | | dependency | | | | | | | | | |
| 28 | 62 | M | Chronic | F2 | 14 | | | YES | | YES | | YES |
| | | | alcoholism | | | | | | | | | |
| 29 | 58 | M | Chronic | F2 | 14 | | | YES | | YES | | YES |
| | | | alcoholism | | | | | | | | | |
| 30 | 40 | M | Chronic | F2 | 13 | | | YES | | YES | | YES |
| | | | alcoholism | | | | | | | | | |
| | | | Tobacco | | | | | | | | | |
| | | | dependency | | | | | | | | | |
| 31 | 29 | M | Videogame | F4 | 10 | | X | | | YES | | YES |
| | | | disorder | | | | | | | | | |
| 32 | 45 | F | Chronic | F2 | 11 | | | YES | X | | X | |
| | | | alcoholism | | | | | | | | | |
| 33 | 27 | M | Cocaine | F4 | 14 | | | YES | | YES | | YES |
| | | | dependency | | | | | | | | | |
| 34 | 25 | M | Chronic | F2 | 10 | | | YES | | YES | | YES |
| | | | alcoholism | | | | | | | | | |
| 35 | 26 | M | Chronic | F2 | 15 | | | YES | | YES | | YES |
| | | | alcoholism | | | | | | | | | |
| | | | Tobacco | | | | | | | | | |
| | | | dependency | | | | | | | | | |
| | | | Drug | | | | | | | | | |
| | | | combination | | | | | | | | | |
| | | | dependency | | | | | | | | | |
| | | | (marijuana, | | | | | | | | | |
| | | | cocaine) | | | | | | | | | |
| 36 | 40 | M | Marijuana | F5 | 10 | | | YES | | YES | | YES |
| | | | dependency | | | | | | | | | |
| 37 | 34 | F | Tobacco | F5 | 10 | | | YES | | YES | | YES |
| | | | dependency | | | | | | | | | |
| | | | Marijuana | | | | | | | | | |
| | | | dependency | | | | | | | | | |
| 38 | 31 | M | Psychostim | F4 | 15 | | | YES | | YES | | YES |
| | | | ulant drug | | | | | | | | | |
| | | | dependency | | | | | | | | | |
| | | | (ecstasy) | | | | | | | | | |
| 39 | 52 | M | Tobacco | F5 | 10 | | | YES | | YES | | YES |
| | | | dependency | | | | | | | | | |
| 40 | 43 | F | Marijuana | F5 | 13 | | | YES | | YES | | YES |
| | | | dependency | | | | | | | | | |
| 41 | 59 | M | Ludopathy | F4 | 10 | | NO | | X | | | YES |
| 42 | 21 | M | Tobacco | F5 | 13 | | | YES | | YES | | YES |
| | | | dependency | | | | | | | | | |
| | | | Cocaine | | | | | | | | | |
| | | | dependency | | | | | | | | | |
| 43 | 36 | F | Drug | F2 | 14 | | | YES | | YES | | YES |
| | | | combination | | | | | | | | | |
| | | | dependency | | | | | | | | | |
| | | | (marijuana, | | | | | | | | | |
| | | | ecstasy, | | | | | | | | | |
| | | | LSD) | | | | | | | | | |
| 44 | 38 | M | Marijuana | F5 | 10 | | | YES | | YES | | YES |
| | | | dependency | | | | | | | | | |
| 45 | 25 | F | Chronic | F2 | 12 | | | YES | | YES | | YES |
| | | | alcoholism | | | | | | | | | |
| | | | Tobacco | | | | | | | | | |
| | | | dependency | | | | | | | | | |
| 46 | 62 | M | Chronic | F2 | 15 | | | YES | | YES | | YES |
| | | | alcoholism | | | | | | | | | |

* Dripping is started at a rate of 5 drops per minute during the first 30 minutes, increasing 5 drops
every 30 minutes until a dripping of 20 drops per minute is reached.
** A patient with CHANGES was considered to be anyone who presented a significant improvement
for their condition in the parameters analyzed.

The importance of collaterally administering intravenous or oral nutritional supplements such as probiotics, vitamins and minerals, and applying Colon Hydrotherapy and Lymphatic Drainage, to intravenous treatment, in order to facilitate the process of assimilation of amino acids, vitamins and probiotics was also found; thereby ensuring proper function of neuroreceptors and amino acid balance.

By promoting daily function of neuroreceptor and amino acid balance, most patients noted a decrease in anxiety and expressed improved mental clarity, which provide to the same a greater opportunity to break free from dependencies to addictive substances.

Colon Hydrotherapy is applied before and during administration of the formulations of the present invention, to ensure cleansing of the bowel and hydration of the body, thereby facilitating detoxification of the organism and a better assimilation of the components of the formulations described herein.

The efficacy of Colon Hydrotherapy in patients previously treated with the formulations of the present invention was evaluated as follows.

A normalization in the frequency of bowel movements was observed. Some patients with mild constipation had 2 and up to 3 bowel movements in 24 hours.

A decrease in meteorism was observed in some patients.

A decrease in post-pandrial abdominal pain and a feeling of fullness were observed in some patients.

An improvement in peristalsis was also observed during daily physical examination.

Weight loss between 300 and 1,200 g was observed.

Some patients reported feeling less irritable.

The decrease in early sensation of satiety was also reported by some patients.

When treated with Colon Hydrotherapy and with the formulations of the present invention, patients showed digestive and systemic benefits.

Regarding to Lymphatic Drainage, this was done manually and was applied two to three times a week before administration of the formulations of the present invention. Its main effect is as an anti-inflammatory and deedematizing, favoring the lymphatic and venous return, thus reducing the interstitial pressure wherewith the tissue acquires better trophic conditions. On the nervous system, it acts as a sedative, analgesic, stimulant and relaxant through the inhibitory effect on the intercalary neuron, which, due to the gentle tactile stimuli, partially reduces the conduction of painful impulses and favors parasympathetic activity. At the same time, it expands thoracic expansion and abdominal respiration with increased lymphatic pumping. It regulates the tone on the striated and smooth muscles, increases the contractile capacity of smooth muscles of lymphatic vessels, thus stimulating the volume of evacuation, stimulates intestinal peristalsis and bladder emptying which results in increased defecation and diuresis; activates the lymphoid organs with an increase in the production of phagocytic cells and antibodies, which are very useful in infectious, autoimmune and tumor processes.

The efficacy of Lymphatic Drainage in patients treated with the formulations of the present invention was evaluated as follows.

Patients who had pain or a feeling of heaviness in the legs improved up to 50%.

The appearance of the skin also improved (especially in those patients with cellulitis data).

A relaxing effect was reported at the limb level and at the systemic level.

Some changes were observed in patients with telangiectasias.

By improving the lymphatic system, circulation and encourage toxins elimination and fluids is favored; and hence the assimilation of the components of the formulations described herein.

At the beginning of administration of the formulations of the present invention, some patients reported some minor nasal congestion, heaviness in the chest or gastrointestinal symptoms, being this behavior normal, since it is a response to the withdrawal syndrome; due to the brain and body are assimilating the nutrients necessary for their detoxification and repair, through nutrients which they lacked for a long time, which required a slowdown in the drip rate. As the treatment with the formulations progressed, the patient was able to receive formulations intravenously at a faster rate. There was evidence of a need for drip rates to start slowly the first day at 5 drops per minute for the first 30 minutes and then increase to 10, 15 and 20 drops per minute every 30 minutes, as tolerated by the patient. The IV should not exceed a drip rate greater than 20 drops per minute, even if the patient shows no signs of intolerance or toxicity at drip rates above this dose.

The results showed that it is essential to guarantee a sufficient and adequate administration of the formulations of the present invention for a sufficient number of hours a day (between 6-8 hours), otherwise the addicted patient will not be prevented from experiencing an important abstinence syndrome while enabling tissue healing and preventing or improving withdrawal symptoms. It was found that a very fast administration or the lack of supplements with vitamins and probiotics may cause amino acids are not assimilated and their accumulation to become toxic for the body. If dripping is too fast (i.e., above 20 drops per minute), the patient will complain of severe nasal congestion, chest pressure, and/or gastrointestinal discomfort. This is resolved within 5 to 20 minutes, depending on each patient, as soon as the drip rate is slowed down or stopped. If dripping is not fast enough to cause the above symptoms, but is still too fast, the patient may experience a headache or feel exhausted, and may experience insomnia. The normal daily dose was determined from 250 ml (half a vial) to 500 ml (one vial) of the formulations contained in 0.9% NaCl solution for intravenous administration.

At the beginning of the treatment, in 7% of the cases (approximately 3 patients), some gastrointestinal discomfort due to drug withdrawal was reported. These discomforts may be caused by the fact that the gastrointestinal system is abundantly filled with neurotransmitters, such as serotonin, which affects peristalsis and other nervous functions. In this sense, it is important to note that the majority of the body's serotonin, around 80% or 90%, is found in the gastrointestinal tract. The concentration of this neurotransmitter can be reduced by stress and it has an influence on the mood, anxiety and happiness. For this reason, gastrointestinal recovery is a fundamental part of treatment in many cases, although not all patients may have digestive symptoms.

Sleep disturbance is a problem that leads to addiction, and it can also be worsened by it. Some cases of insomnia have been reported, which is due to the fact that during the treatment the nervous system and the physiological elements of the sleep cycle are being repaired.

The administration of formulations of the present invention proved its efficacy in people suffering from simple depression or brain fog who do not have a significant history of drug use, or who only want to improve mental acuity. In this regard, the formulation comprising L-threonine in the range of 1.0-3.0 mg, glycine in the range of 1.0-3.0 mg, L-phenylalanine in the range of 1.0-3.0 mg, D,L-phenylalanine in the range of 2.0-5.0 mg, L-alanyl/L-glutamine in the range of 2.0-5.0 mg, NAD in the range of 500-1000 mg and

MgCl₂ in the range of 400-600 mg, favors brain impulse, and hence logical thinking and mental clarity. For people without a history of drug use, treatment with this formulation for five days was sufficient to obtain the expected results.

In the case of formulation comprising L-threonine in the range of 1.0-3.0 mg, glycine in the range of 1.0-3.0 mg, L-phenylalanine in the range of 1.0-3.0 mg, D,L-phenylalanine in the range of 2.0-5.0 mg, L-alanyl/L-glutamine in the range of 2.0-5.0 mg, NAD in the range of 700-1200 mg and MgCl₂ in the range of 400-600 mg, it was observed that it specifically favors recovery from intoxication and withdrawal syndrome in patients with addiction to alcohol and opiates.

The formulation that specifically comprises L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 300-500 mg and MgCl₂ in the range of 400-600 mg, favors recovery in patients with addiction to benzodiazepines, antidepressants, hypnotics, antipsychotics, anticonvulsants, muscle relaxants.

Administration of the formulation comprising L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 200-300 mg and MgCl₂ in the range of 300-600 mg, favors recovery in patients with cocaine, methamphetamine and antipsychotic addiction; in addition, a recovery was also observed in patients with some addictive behaviors (pornography, games, electronic devices).

Recovery in patients with addiction to tobacco, marijuana and other types of cannabinoids was observed when treated with the formulation comprising L-threonine in the range of 0.5-1.0 mg, glycine in the range of 0.5-1.0 mg, L-phenylalanine in the range of 0.5-1.0 mg, D,L-phenylalanine in the range of 1.0-2.0 mg, L-alanyl/L-glutamine in the range of 1.0-2.0 mg, NAD in the range of 200-400 mg and MgCl₂ in the range of 300-600 mg.

No adverse effects were observed for the administration of the formulations of the present invention, nor pharmacological interactions. This is because such formulations are inherently safe, as they are composed entirely of amino acids -which are the building blocks of proteins-, vitamins, minerals, and co-enzymes.

The formulations of the present invention allowed the recovery of patients with addictions to psychotropic medications, illicit drugs, tobacco and alcohol, including narcotics, benzodiazepines, marijuana, cocaine, methamphetamines, antidepressants and antipsychotics, due to the balanced combination of amino acids, peptides, coenzyme NAD and magnesium chloride at low concentrations.

The inventors of the present invention found that said combinations potentiated the effects of each of the components which separately would not have had the same result.

The function of amino acids combined with other nutrients such as vitamins, minerals and the B complex, further enhance brain functioning.

The inventors of the present invention consider that there is a synergy of the components in the present formulations, mainly given by magnesium chloride, since it is known that this ion influences the absorption of aromatic amino acids in the brain.

### REFERENCES

1. Alhulia S. Intoxicación por drogas de abuso. Uninet. 2014.

2. Aranda Pilar, Planells Elena y LLopis Juan. Magnesio. Scientific Communication: Art o Technique? Ars Pharmaceutica. 2000; 41(1): 91-100.

3. Avila Ariel, Laurent Nguyen, and Rigo Jean-Michel. Glycine receptors and brain development, Cell Neurosci. 2013; 7: 184.

4. Bonet A, Grau T. La glutamina, un aminoácido casi indispensable en el enfermo crítico. Med Intensiva. 2007; 31: 402-406.

5. Cambridge Dictionaries Online. “Amino Acid”. Cambridge University Press. 2015. Retrieved 3 Jul. 2015.

6. Castro-Marrero J, Cordero M D, Segundo M J, et al. Does Oral Coenzyme Q10Plus NADH Supplementation Improve Fatigue and Biochemical Parameters in Chronic Fatigue Syndrome Antioxidants & Redox Signaling. 2015; 22(8):679-685.

7. Clark D, Pollock N, Bukstein O, et al. Gender and comorbid psychopathology in adolescents with alcohol dependence. J am acad child adolesc psychiatry. 1997; 36:1195.

8. Daini S, Tonioni F, Barra A, Lai C, Lacerenza R, Sgambato A, Bria P, Cittadini A. Serum magnesium profile in heroin addicts according to psychiatric comorbidity. Magnes Res. 2006; 19:162-6.

9. Daly J W, Fredholm B B. Caffeine an atypical drug of dependence. Drug Alcohol Depend. 1998; 51:199-206.

10. Diana M, Peana AT, Sirca D, Lintas A, Melis M, Enrico P. Crucial role of acetaldehyde in alcohol activation of the mesolimbic dopamine system. Ann N Y Acad Sci. 2008; 1139:307-17.

11. Foddai M, Dosia G, Spiga S, Diana M. Acetaldehyde increases dopaminergic neuronal activity in the VTA. Neuropsychopharmacology. 2004; 29:530-6.

12. Friedman, M. Nutritional y toxicological consequences of food processing, New York, Plenum Press. 1991; p. 447-481.

13. Garcia de Lorenzo A. Glutamina, puesta al dia y controversias. Nutr Clin en Medicina. 2008; 1: 23-35.

14. Gass J T, Olive M F. Glutamatergic substrates of drug addiction and alcoholism. Biochem Pharmacol. 2008; 75:218-65.

15. Gianoulakis C. Endogenous opioids and addiction to alcohol and other drugs of abuse. Curr Top Med Chem. 2009; 9:999-1001.

16. Goldstein R Z, Volkow N D. Dysfunction of the prefrontal cortex in addiction: neuroimaging findings and clinical implications. Nat Rev Neurosci. 2011; 12(11):652-669.

17. Gray J A, Moran P M, Grigoryan G, Peters SL, Young A M, Joseph M H. Latent inhibition: the nucleus accumbens connection revisited. Behav Brain Res. 1997; 88:27-34.

18. Herz A. Endogenous opioid systems and alcohol addiction. Psychopharmacology (Berl). 1997; 129:99-111.

19. Hoane Michael R. Magnesium in the Central Nervous System.

Chapter 7. The role of magnesium therapy in learning and memory. 2011; Pages 115-124.

20. Hoffman B B. Cathecolamines, sympathomimetic drugs, and adrenergic receptor antagonists, In: Goodman & Gilman's The Pharmacological Basis of Therapeutics (Hardman J G, Limbird L E, eds.) Tenth International Edition, New York, Mc Graw Hill. 2001; 215-68.

21. Hughes J R, Oliveto A H, Helzer J E, Higgins S T, Bickel W K. Should caffeine abuse, dependence, or withdrawal be added to DSM IV and ICD 10? Am J Psychiatry. 1992; 149:33-40.

22. Humiston J. “Treatment of Drug and Alcohol Dependence and Chronic Pain with Intravenous Amino Acids.” Meeting of the Int'l College of Integrative Medicine, Dearborn, Michigan, 25 Sep. 2014

23. Institute of Medicine. “Protein and Amino Acids”. Dietary Reference Intakes for Energy, Carbohydrates, Fiber, Fat, Fatty Acids, Cholesterol, Protein, and Amino Acids. Washington, D.C.: The National Academies Press. 2002; pp. 589-768.

24. IUPAC-IUB Joint Commission on Biochemical Nomenclature “Nomenclature and Symbolism for Amino Acids and Peptides. 1983. Archived from the original on 9 Oct. 2008. Retrieved 5 Mar. 2018.

25. Jaramillo-Magaña José J. Metabolismo cerebral. ANESTESIOLOGÍA EN NEUROCIRUGÍA. 2013; 36(1) 183-185.

26. Kantak K M, Edwards M A, O'Connor T P. Modulation of the discriminative stimulus and rate altering effects of cocaine by competitive and noncompetitive N-methyl D aspartate antagonists. Pharmacol Biochem Behav. 1998; 59:159-69.

27. Karakiewicz B, Kozielec T, Chlubek D, Noceri I, Starczewski A, Brodowska A, Laszczylnska M. Serum magnesium concentration in drug addicted patients. Magnes Res. 2007; 20:55-7.

28. Kaya Mehmet and Ahishali Bulent. Magnesium in the Central Nervous System. Chapter 9. The role of magnesium in edema and blood brain barrier disruption. 2011; Pages 135-144.

29. Kelly T , Daley D . Integrated treatment of substance use and psychiatric disorders. Soc Work Public Health. 2013; 8(0):388-406.

30. Kirkland Anna E., Sarlo Gabrielle L. and Holton Kathleen F. The Role of Magnesium in Neurological Disorders. Nutrients. 2018(10): 730.

31. Koob G , Le Moal M. Drug addiction, dysregulation of reward, and allostasis. Neuropsychopharmacology. 2001; 24:97-129.

32. Kosten T . Addiction as a brain disease. Am J Psychiatry. 1998; 155:711-13.

33. Margolin A, Kantak K, Copenhaver M, Avant S S K. A preliminary controlled investigation of magnesium L-aspartate hydrochloride for illicit cocaine and opiate use in methadone maintained patients. J Addict Dis. 2003; 22:49-61.

34. McLellan A T, Lewis D C, O'Brien C P, Kleber. Drug dependence, a chronic medical illness. JAMA. 2000; 284:1689-95.

35. Mendez M, Leriche M, Calva J C. Acute etanol administration differentially modulates mu opioid receptors in the rat meso accumbens and mesocortical pathways. Brain Res Mol Brain Res. 2001; 94:148-56.

36. Miguel-Hidalgo J J. The role of glial cells in drug abuse. Curr Drug Abuse. 2009; Rev 2:72-82.

37. Morán Chamorro I, Baldirá Martinez de Irujo J, Marruecos-Sant L, Nogué Xarau S. Anfetaminas y drogas de sintesis. In: Difusión G, editor. Toxicologia clinica. Madrid; 2011. p. 499-510.

38. Morán Chamorro I, Baldirá Martinez de Irujo J, Marruecos-Sant L, Nogué Xarau S. Intoxicación aguda por derivados del cannabis. In: Difusión G, editor. Toxicologia clinica. Madrid; 2011. p. 463-7.

39. Morán Chamorro I, Baldirá Martinez de Irujo J, Marruecos-Sant L, Nogué Xarau S. Intoxicación aguda por bebidas energizantes (a base de taurina, inositol y cafeina). In: Difusión G, editor. Toxicologia clinica. Madrid; 2011. p. 535-8

40. Morán Chamorro I, Baldirá Martinez de Irujo J, Marruecos-Sant L, Nogué Xarau S. Intoxicación por opiáceos y opioides. In: Difusión G, editor. Toxicologia clinica. Madrid; 2011. p. 483-95.

41. Morán Chamorro I, Baldirá Martinez de Irujo J, Marruecos-Sant L, Nogué Xarau S. Intoxicación y sobredosis por cocaina. In: Difusión G, editor. Toxicologia clinica. Madrid; 2011. p. 473-80.

42. Nechifor M, Chelarescu D, Ciubotariu D. The influence of magnesium induced stimulation of the reward system. Magnesium Res. 2010; 23:41-7.

43. Nechifor M, Chelarescu D, Mândreci I, Cartas N. Magnesium influence on nicotine pharmaco dependence and smoking. Magnes Res. 2004; 17:176-81.

44. Nechifor M, Chelarescu D, Miftode M. Magnesium influence on morphine induced pharmacodependence in rats. Magnes Res. 2004; 17:7-13.

45. Nechifor Mihai. Magnesium in the Central Nervous System. Chapter 24 Magnesium in drug abuse and addiction. 2011; Pages 331-342.

46. Nelson, David L.; Cox, Michael M., Principles of Biochemistry (4th ed.), New York: W. H. Freeman, (2005) ISBN 0-7167-4339-6.

47. Niemela J E, Cecco S A, Rehak N N, Elin R J. The effect of smoking on the serum ionized magnesium concentration is method dependent. Arch Pathol Lab Med. 1997; 121:1087-92.

48. Ogawa N, Ueki H. Clinical importance of caffeine dependence and abuse. Psychiatry Clin Neurosci. 2007; 61:263-8.

49. Rodriguez F D, Bardaji E, Trayno Jr R. Differential effects of Mg²⁺ and other divalent cation on the binding of tritiated opioid ligands. J Neurochem. 1992; 591:467-72.

50. Ruiz Martinez M, Gil Extremera B, Maldonaldo M D, Cantero-Hinojosa J, Moreno-Abadia V. Trace elements in drug addicts. Klin Wochenschr. 1990; 68:507-11.

51. Substance Abuse and Mental Health Services Administration. Mental and Substance Use Disorders. https://www.samhsa.gov/disorders. Published Jun. 20, 2014.

52. Sutoo D, Akiyama K. Effect of magnesium on calcium dependent brain function that prolongs etanol induced sleeping time in mice. Neurosci Lett. 2000; 204:5-8.

53. Tonioni F, Martinotti G, Barra A, Martinelli D, Autullo G, Rinaldi C, Tedeschi C, Janiri L, Bria P. Cocaine use disorders and serum magnesium profile, Neuropsychobiology. 2009; 53:159-64.

54. Trulson M E, Arasteh K. Morphine increases the activity of midbrain dopamine neurons in vitro. Eur J Pharmacol. 1985; 114:105-9.

55. Yablon Lisa A. and Mauskop Alexander. Magnesium in the Central Nervous System. Chapter 8. Magnesium in headache. 2011; Pages 125-134.

56. https://www.mayoclinic.org/healthy-lifestyle/consumer-health/expert-answers/colon-cleansing/faq-20058435

57. file:///C:/Users/Styku/Downloads/Dialnet-BasesTerapeuticasDelLinfodrenajeManualDeVodder-4956302.pd

1. Pharmaceutical formulation comprising:
L-threonine;
Glycine;
L-phenylalanine;
DL-phenylalanine;
Glutamine;
NAD; and
Magnesium.

2. The pharmaceutical formulation according to claim 1, wherein the components of the formulation are solubilized in a 0.9% sodium chloride (NaCl) solution.

3. The formulation according to claim 1, wherein the glutamine is preferably in the form of a L-alanine/L-glutamine dipeptide.

4. The formulation according to claim 1, wherein the preferred magnesium is in the form of magnesium chloride.

5. The formulation according to claims 1-4, wherein said formulation is adapted to be administrable intravenously.

6. The formulation according to claims 1-5, for use in improving brain impulse and mental clarity in a subject.

7. The formulation according to claims 1-5, for use in the treatment of addictions in a subject in need thereof.

8. The formulation according to claim 7, wherein the addictions are selected from the group consisting of consumption of psychotropic medications, illicit drugs, tobacco and alcohol, including narcotics, benzodiazepines, marijuana, cocaine, methamphetamines, antidepressants, antipsychotics, or alcohol.

9. The formulation according to claims 6-8, wherein the formulation is optionally administrable with commercially available vitamins, probiotics and minerals.

10. The formulation according to claims 6-8, wherein the formulation is optionally administrable with Colon Hydrotherapy and Lymphatic Drainage.

11. The formulation according to claim 10, wherein the Colon Hydrotherapy is applied before and during administration of the formulation.

12. The formulation according to claim 10, wherein the Lymphatic Drainage is applied manually, two to three times a week before administration of the formulation.

13. The formulation according to claims 6-8, wherein the formulation can be administered intravenously by drip to the subject.

14. The formulation according to claim 13, wherein the drip rate is 5 to 20 drops per minute.

15. The formulation according to claims 6-8, wherein the formulation is administrable in a 500 ml vial presentation.

16. The formulation according to claims 6-8, wherein the formulation can be administered for five to fifteen days uninterruptedly.

17. The formulation according to claims 1-5, wherein L-threonine is in the range of 0.5-3.0 mg, glycine in the range of 0.5-3.0 mg, L-phenylalanine in the range of 0.5-3.0 mg, DL-phenylalanine in the range of 1.0-5.0 mg, L-alanyl/L-glutamine in the range of 1.0-5.0 mg, NAD in the range of 200-1200 mg, and MgCl₂ in the range of 300-600 mg.

18. The formulation according to claim 17, for use in improving brain impulse and mental clarity in a subject.

19. The formulation according to claim 17, for use in the treatment of addictions in a subject in need thereof.

20. The formulation according to claim 19, wherein the addictions are selected from the group consisting of consumption of psychotropic medications, illicit drugs, tobacco and alcohol, including narcotics, benzodiazepines, marijuana, cocaine, methamphetamines, antidepressants, antipsychotics, or alcohol.

21. The formulation according to claims 18-20, wherein the formulation is optionally administrable with commercially available vitamins, probiotics and minerals.

22. The formulation according to claims 18-20, wherein the formulation is optionally administrable with Colon Hydrotherapy and Lymphatic Drainage.

23. The formulation according to claim 22, wherein the Colon Hydrotherapy is applied before and during administration of the formulation.

24. The formulation according to claim 22, wherein the Lymphatic Drainage is applied manually, two to three times a week before administration of the formulation.

25. The formulation according to claims 18-20, wherein the formulation can be administered intravenously by drip to the subject.

26. The formulation according to claim 25, wherein the drip rate is 5 to 20 drops per minute.

27. The formulation according to claims 18-20, wherein the formulation is administrable in a 500 ml vial presentation.

28. The formulation according to claims 18-20, wherein the formulation is administrable for five to fifteen days uninterruptedly.

29. The formulation according to claims 1-5, wherein L-threonine is in the range of 1.0-3.0 mg, glycine in the range of 1.0-3.0 mg, L-phenylalanine in the range of 1.0-3.0 mg, DL-phenylalanine in the range of 2.0-5.0 mg, L-alanyl/L-glutamine in the range of 2.0-5.0 mg, NAD in the range of 500-1000 mg and MgCl₂ in the range of 400-600 mg.

30. The formulation according to claim 29, for use in the treatment of simple depression or brain fog in subjects who do not have a significant history of drug use, or who only want to improve mental acuity.

31. The formulation according to claim 30, wherein the formulation is optionally administrable with commercially available vitamins, probiotics and minerals.

32. The formulation according to claim 30, wherein the formulation is optionally administrable with Colon Hydrotherapy and Lymphatic Drainage.

33. The formulation according to claim 32, wherein the Colon Hydrotherapy is applied before and during administration of the formulation.

34. The formulation according to claim 32, wherein the Lymphatic Drainage is applied manually, two to three times a week before administration of the formulation.

35. The formulation according to claim 30, wherein the formulation is administrable intravenously by drip to the subject.

36. The formulation according to claim 35, wherein the drip rate is 5 to 20 drops per minute.

37. The formulation according to claim 30, wherein the formulation is administrable in a 500 ml vial presentation.

38. The formulation according to claim 30, wherein the formulation is administrable for five days uninterruptedly.

39. The formulation according to claims 1-5, wherein L-threonine is in the range of 1.0-3.0 mg, glycine in the range of 1.0-3.0 mg, L-phenylalanine in the range of 1.0-3.0 mg, DL-phenylalanine in the range of 2.0-5.0 mg, L-alanyl/L-glutamine in the range of 2.0-5.0 mg, NAD in the range of 700-1200 mg and MgCl₂ in the range of 400-600 mg.

40. The formulation according to claim 39, for use in the recovery of intoxication and withdrawal syndrome in subjects with addiction to alcohol and opiates.

41. The formulation according to claim 40, wherein the formulation is optionally administrable with commercially available vitamins, probiotics and minerals.

42. The formulation according to claim 40, wherein the formulation is optionally administrable with Colon Hydrotherapy and Lymphatic Drainage.

43. The formulation according to claim 42, wherein the Colon Hydrotherapy is applied before and during administration of the formulation.

44. The formulation according to claim 42, wherein the Lymphatic Drainage is applied manually, two to three times a week before administration of the formulation.

45. The formulation according to claim 40, wherein the formulation is administrable intravenously by drip to the subject.

46. The formulation according to claim 45, wherein the drip rate is 5 to 20 drops per minute.

47. The formulation according to claim 40, wherein the formulation is administrable in a 500 ml vial presentation.

48. The formulation according to claim 40, wherein the formulation is administrable for ten to fifteen days uninterruptedly.

49. The formulation according to claims 1-5, wherein L-threonine is in the range of 0.5-1.0 mg, glycine in the range of 0.5-1.0 mg, L-phenylalanine in the range of 0.5-1.0 mg, DL-phenylalanine in the range of 1.0-2.0 mg, L-alanyl/L-glutamine in the range of 1.0-2.0 mg, NAD in the range of 300-500 mg and MgCl₂ in the range of 400-600 mg.

50. The formulation according to claim 49, for use in the recovery of subjects addicted to benzodiazepines, antidepressants, hypnotics, antipsychotics, anticonvulsants, muscle relaxants.

51. The formulation according to claim 50, wherein the formulation is optionally administrable with commercially available vitamins, probiotics and minerals.

52. The formulation according to claim 50, wherein the formulation is optionally administrable with Colon Hydrotherapy and Lymphatic Drainage.

53. The formulation according to claim 52, wherein the Colon Hydrotherapy is applied before and during administration of the formulation.

54. The formulation according to claim 52, wherein the Lymphatic Drainage is applied manually, two to three times a week before administration of the formulation.

55. The formulation according to claim 50, wherein the formulation is administrable intravenously by drip to the subject.

56. The formulation according to claim 55, wherein the drip rate is 5 to 20 drops per minute.

57. The formulation according to claim 50, wherein the formulation is administrable in a 500 ml vial presentation.

58. The formulation according to claim 50, wherein the formulation is administrable for ten to fifteen days uninterruptedly.

59. The formulation according to claim 58, wherein the formulation is administrable for a further three to five days to subjects addicted to benzodiazepines.

60. The formulation according to claims 1-5, wherein L-threonine is in the range of 0.5-1.0 mg, glycine in the range of 0.5-1.0 mg, L-phenylalanine in the range of 0.5-1.0 mg, DL-phenylalanine in the range of 1.0-2.0 mg, L-alanyl/L-glutamine in the range of 1.0-2.0 mg, NAD in the range of 200-300 mg and MgCl₂ in the range of 300-600 mg.

61. The formulation according to claim 60, for use in the recovery of subjects addicted to cocaine, methamphetamines and antipsychotics; and in the recovery of subjects with some addictive behaviors (pornography, games, electronic devices).

62. The formulation according to claim 61, wherein the formulation is optionally administrable with commercially available vitamins, probiotics and minerals.

63. The formulation according to claim 61, wherein the formulation is optionally administrable with Colon Hydrotherapy and Lymphatic Drainage.

64. The formulation according to claim 63, wherein the Colon Hydrotherapy is applied before and during administration of the formulation.

65. The formulation according to claim 63, wherein the Lymphatic Drainage is applied manually, two to three times a week before administration of the formulation.

66. The formulation according to claim 61, wherein the formulation is administrable intravenously by drip to the subject.

67. The formulation according to claim 66, wherein the drip rate is 5 to 20 drops per minute.

68. The formulation according to claim 61, wherein the formulation is administrable in a 500 ml vial presentation.

69. The formulation according to claim 61, wherein the formulation is administrable for ten to fifteen days uninterruptedly.

70. The formulation according to claims 1-5, wherein L-threonine is in the range of 0.5-1.0 mg, glycine in the range of 0.5-1.0 mg, L-phenylalanine in the range of 0.5-1.0 mg, DL-phenylalanine in the range of

1. 0-2.0 mg, L-alanyl/L-glutamine in the range of 1.0-2.0 mg, NAD in the range of 200-400 mg and MgCl₂ in the range of 300-600 mg.

71. The formulation according to claim 70, for use in the recovery of subjects addicted to tobacco, marijuana and other types of cannabinoids.

72. The formulation according to claim 71, wherein the formulation is optionally administrable with commercially available vitamins, probiotics and minerals.

73. The formulation according to claim 71, wherein the formulation is optionally administrable with Colon Hydrotherapy and Lymphatic Drainage.

74. The formulation according to claim 73, wherein the Colon Hydrotherapy is applied before and during administration of the formulation.

75. The formulation according to claim 73, wherein the Lymphatic Drainage is applied manually, two to three times a week before administration of the formulation.

76. The formulation according to claim 71, wherein the formulation is administrable intravenously by drip to the subject.

77. The formulation according to claim 76, wherein the drip rate is 5 to 20 drops per minute.

78. The formulation according to claim 71, wherein the formulation is administrable in a 500 ml vial presentation.

79. The formulation according to claim 71, wherein the formulation is administrable for ten to fifteen days uninterruptedly.

80. A method of treatment for improving brain impulse and mental clarity, which comprises administering to a subject a therapeutically effective amount of the formulation of claim 17.

81. An addiction treatment method, which comprises administering to a subject a therapeutically effective amount of claim 17.

82. The method according to claim 81, wherein the addictions are selected from the group consisting of consumption of psychotropic medications, illicit drugs, tobacco and alcohol, including narcotics, benzodiazepines, marijuana, cocaine, methamphetamines, antidepressants, antipsychotics, or alcohol.

83. The method according to claims 80-82, wherein the formulation is optionally administered with commercially available vitamins, probiotics and minerals.

84. The treatment method of claims 80-82, wherein the formulation is optionally administered with Colon Hydrotherapy and Lymphatic Drainage.

85. The method according to claim 84, wherein the Colon Hydrotherapy is applied before and during the administration of the formulation.

86. The method according to claim 84, wherein the Lymphatic Drainage is applied manually, two to three times a week before administration of the formulation.

87. The method according to claims 80-82, wherein the formulation is administered intravenously by drip to the subject.

88. The method according to claim 87, wherein the drip rate is 5 to 20 drops per minute.

89. The method according to claims 80-82, wherein the formulation is administered in a 500 ml vial presentation.

90. The method according to claims 80-82, wherein the formulation is administered for five to fifteen days uninterruptedly.

91. A method of treatment for simple depression or brain fog in subjects who do not have a significant history of drug use, or who only want to improve mental acuity, which comprises administering to said subject a therapeutically effective amount of the formulation of claim 29.

92. The method according to claim 91, wherein the formulation is optionally administered with commercially available vitamins, probiotics and minerals.

93. The method according to claim 91, wherein the formulation is optionally administered with Colon Hydrotherapy and Lymphatic Drainage.

94. The method according to claim 93, wherein the Colon Hydrotherapy is applied before and during the administration of the formulation.

95. The method according to claim 93, wherein the Lymphatic Drainage is applied manually, two to three times a week before administration of the formulation.

96. The method according to claim 91, wherein the formulation is administered intravenously by drip to the subject.

97. The method according to claim 96, wherein the drip rate is 5 to 20 drops per minute.

98. The method according to claim 91, wherein the formulation is administered in a 500 ml vial presentation.

99. The method according to claim 91, wherein the formulation is administered for five days uninterruptedly.

100. A method of treatment for the recovery of intoxication and withdrawal syndrome in subjects with addiction to alcohol and opiates, which comprises administering to said subjects a therapeutically effective amount of the formulation of claim 39.

101. The method according to claim 100, wherein the formulation is optionally administered with commercially available vitamins, probiotics and minerals.

102. The method according to claim 100, wherein the formulation is optionally administered with Colon Hydrotherapy and Lymphatic Drainage.

103. The method according to claim 102, wherein the Colon Hydrotherapy is applied before and during the administration of the formulation.

104. The method according to claim 102, wherein the Lymphatic Drainage is applied manually, two to three times a week before administration of the formulation.

105. The method according to claim 100, wherein the formulation is administered intravenously by drip to the subject.

106. The method according to claim 105, wherein the drip rate is 5 to 20 drops per minute.

107. The method according to claim 100, wherein the formulation is administered in a 500 ml vial presentation.

108. The method according to claim 100, wherein the formulation is administered for ten to fifteen days uninterruptedly.

109. A method of treatment for the recovery of subjects addicted to benzodiazepines, antidepressants, hypnotics, antipsychotics, anticonvulsants, muscle relaxants, which comprises administering to said subjects a therapeutically effective amount of the formulation of claim 49.

110. The method according to claim 109, wherein the formulation is optionally administered with commercially available vitamins, probiotics and minerals.

111. The method according to claim 109, wherein the formulation is optionally administered with Colon Hydrotherapy and Lymphatic Drainage.

112. The method according to claim 111, wherein the Colon Hydrotherapy is applied before and during the administration of the formulation.

113. The method according to claim 111, wherein the Lymphatic Drainage is applied manually, two to three times a week before administration of the formulation.

114. The method according to claim 109, wherein the formulation is administered intravenously by drip to the subject.

115. The method according to claim 114, wherein the drip rate is 5 to 20 drops per minute.

116. The method according to claim 109, wherein the formulation is administered in a 500 ml vial presentation.

117. The method according to claim 109, wherein the formulation is administered for ten to fifteen days uninterruptedly.

118. The method according to claim 117, wherein the formulation is administered for three to five more days to subjects addicted to benzodiazepines.

119. A treatment method for the recovery of subjects with addiction to cocaine, methamphetamines and antipsychotics; and for the recovery of subjects with some addictive behaviors (pornography, games, electronic devices), which comprises administering to said subjects a therapeutically effective amount of the formulation of claim 60.

120. The method according to claim 119, wherein the formulation is optionally administered with commercially available vitamins, probiotics and minerals.

121. The method according to claim 119, wherein the formulation is optionally administered with Colon Hydrotherapy and Lymphatic Drainage.

122. The method according to claim 121, wherein the Colon Hydrotherapy is applied before and during the administration of the formulation.

123. The method according to claim 121, wherein the Lymphatic Drainage is applied manually, two to three times a week before administration of the formulation.

124. The method according to claim 119, wherein the formulation is administered intravenously by drip to the subject.

125. The method according to claim 124, wherein the drip rate is 5 to 20 drops per minute.

126. The method according to claim 119, wherein the formulation is administered in a 500 ml vial presentation.

127. The method according to claim 119, wherein the formulation is administered for ten to fifteen days uninterruptedly.

128. A method of treatment for the recovery of subjects addicted to tobacco, marijuana and other types of cannabinoids, which comprises administering to said subjects a therapeutically effective amount of the formulation of claim 70.

129. The method according to claim 128, wherein the formulation is optionally administered with commercially available vitamins, probiotics and minerals.

130. The method according to claim 128, wherein the formulation is optionally administered with Colon Hydrotherapy and Lymphatic Drainage.

131. The method according to claim 130, wherein the Colon Hydrotherapy is applied before and during the administration of the formulation.

132. The method according to claim 130, wherein the Lymphatic Drainage is applied manually, two to three times a week before administration of the formulation.

133. The method according to claim 128, wherein the formulation is administered intravenously by drip to the subject.

134. The method according to claim 133, wherein the drip rate is 5 to 20 drops per minute.

135. The method according to claim 128, wherein the formulation is administered in a 500 ml vial presentation.

136. The method according to claim 128, wherein the formulation is administered for ten to fifteen days uninterruptedly.
